(19) United States
(12) Patent Application Publication
(10) Pub. No.: US 2023/0104356 A1
(43) Pub. Date: Apr. 6, 2023

(54) MODEL DRIVEN SUB-SYSTEM FOR DESIGN AND EXECUTION OF EXPERIMENTS

(71) Applicant: Tata Consultancy Services Limited, Mumbai (IN)

(72) Inventors: ARPIT VISHWAKARMA, Pune (IN); PRASENJIT DAS, Kolkata (IN); PURUSHOTTHAM GAUTHAM BASAVARSU, Pune (IN); SREEDHAR SANNAREDDY REDDY, Pune (IN); AMOL DILIP JOSHI, Pune (IN)

(73) Assignee: Tata Consultancy Services Limited, Mumbai (IN)

(21) Appl. No.: 17/905,038

(22) PCT Filed: Mar. 27, 2021
(86) PCT No.: PCT/IN2021/050320

§ 371 Date: Aug. 25, 2022

(30) Foreign Application Priority Data
Mar. 27, 2020 (IN) 202021013527

(51) Int. Cl. G16B 50/10; G16B 50/30

(52) U.S. Cl. CPC G16B 50/10; G16B 50/30

(57) ABSTRACT

All the model-driven systems may not have capability to perform designing and execution of experiments, which limits functionality of such model-driven systems. The disclosure herein generally relates to Design of Experiments (DOE), and, more particularly, to a model driven sub-system for design and execution of experiments. The sub-system when plugged into the model driven system, uses legacy components as well components of the sub-system to perform designing and execution of the design of experiments.

### CROSS-REFERENCE TO RELATED APPLICATIONS AND PRIORITY

The present application claims priority to India Patent Application No. 202021013527, filed before Indian Patent Office on Mar. 27, 2020. Entire contents of the aforementioned application are incorporated herein by reference.

### TECHNICAL FIELD

The disclosure herein generally relates to Design of Experiments (DOE), and, more particularly, to a model driven sub-system for design and execution of experiments.

### BACKGROUND

Design of Experiments (DOE) is a field of science which deals with planning, conducting, analyzing, and interpreting controlled tests to evaluate factors that control value of a parameter of a group of parameters. A system that performs the DOE need to be capable of analyzing data so as to understand relationship between a process and various parameters. For example, consider a task that involves multiple variables. Change in any or all of these variables result in change in any variables that are dependent on these variables, and in turn on final results/outputs generated. During the DOE of the task, such variables, dependency of one or more other variables, and so on are defined, such that an intended final result can be obtained.

However, most of the times the systems that originally generate or store the data may not be able to perform the data processing for the DOE. As a result, the data may have to be transferred to an external storage medium or one or more external systems having data processing capability to perform the DOE. Further, the external systems may have to be given rights to access the data, which may cause data security issues.

### SUMMARY

Embodiments of the present disclosure present technological improvements as solutions to one or more of the above-mentioned technical problems recognized by the inventors in conventional systems. For example, in one embodiment, a model driven sub-system for design and execution of experiments consisting of a digital workflow with one or more in-silico experiments in a model-driven system is provided. The model driven sub-system includes one or more hardware processors, one or more communication interfaces, and one or more memory storing a plurality of instructions. The plurality of instructions when executed cause the one or more hardware processors to define design of an experiment, and generate a result for the defined design of the experiment, by executing the design of the experiment. Defining the design of experiment includes selecting a system process for the experiment. Further, a functional model for the selected system process is created if the functional model does not already exist. Further, each functional parameter is mapped with corresponding ontology parameters. Further, a meta-design space is initialized for the functional model. Further, the experiment is created from the functional model, wherein a plurality of experiment parameters of the experiment conform to the functional parameters of the functional model. The experiment parameters are attached with the functional parameters, and then an input generator and a distribution generator are selected for the design of the experiment.

In another aspect, a processor implemented method for design and execution of experiments consisting of a digital workflow with one or more in-silico experiments in a model-driven system is provided. The method includes defining design of an experiment, and generating a result for the defined design of the experiment by executing the design of the experiment. Defining the design of experiment includes selecting a system process for the experiment. Further, a functional model for the selected system process is created if the functional model does not already exist. Further, each functional parameter is mapped with corresponding ontology parameters. Further, a meta-design space is initialized for the functional model. Further, the experiment is created from the functional model, wherein a plurality of experiment parameters of the experiment conform to the functional parameters of the functional model. The experiment parameters are attached with the functional parameters, and then an input generator and a distribution generator are selected for the design of the experiment.

The non-transitory computer readable medium initially defines design of an experiment via one or more hardware processors. The non-transitory computer readable medium further generates a result for the defined design of the experiment by executing the design of the experiment. Defining the design of experiment by the non-transitory computer readable medium involves the following steps: Initially a system process is selected for the experiment. Further, a functional model for the selected system process is created if the functional model does not already exist. Further, each functional parameter is mapped with corresponding ontology parameters. Further, a meta-design space is initialized for the functional model. Further, the experiment is created from the functional model, wherein a plurality of experiment parameters of the experiment conform to the functional parameters of the functional model. The experiment parameters are attached with the functional parameters, and then an input generator and a distribution generator are selected for the design of the experiment. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles:

FIG. 1 illustrates an exemplary sub-system for design and execution of experiments, according to some embodiments of the present disclosure.

FIG. 2 is a high-level flow diagram illustrating steps involved in the process of design of experiments, by the sub-system of FIG. 1, according to some embodiments of the present disclosure.

FIGS. 3A, and 3B (collectively referred to as FIG. 3) illustrates a flow diagram depicting steps involved in the process of defining a Design of Experiment (DOE), using the sub-system of FIG. 1, in accordance with some embodiments of the present disclosure.

FIGS. 4A, 4B, and 4C (collectively referred to as FIG. 4) is a flow diagram depicting steps involved in the process of executing the DOE, using the sub-system of FIG. 1, according to some embodiments of the present disclosure.

FIGS. 5A, 5B, and 5C are example architectures of a data model used by the sub-system of FIG. 1, in accordance with some embodiments of the present disclosure.

### DETAILED DESCRIPTION OF EMBODIMENTS

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments. It is intended that the following detailed description be considered as exemplary only, with the true scope being indicated by the following claims.

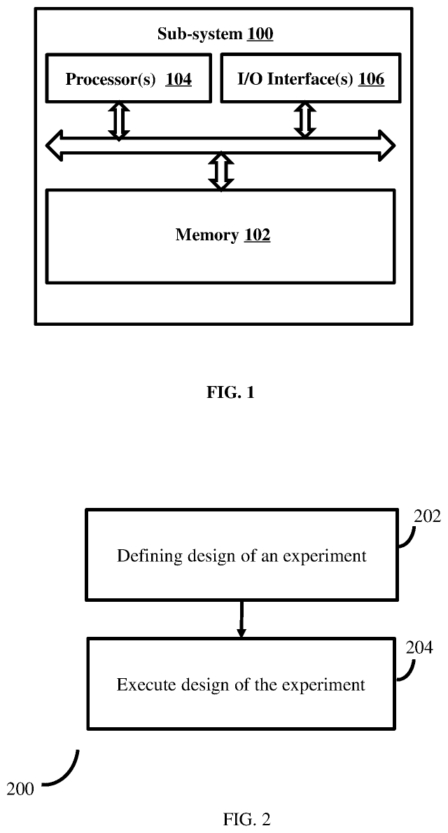

Referring now to the drawings, and more particularly to FIG. 1 through FIG. 5C, where similar reference characters denote corresponding features consistently throughout the figures, there are shown preferred embodiments and these embodiments are described in the context of the following exemplary system and/or method.

FIG. 1 illustrates an exemplary sub-system for design and execution of experiments, according to some embodiments of the present disclosure. The sub-system 100 is implemented in such a way that it can be plugged into a model-driven system that lacks capability to perform the design and execution of experiments, so as to enable the model-driven system to design and evaluate design of experiment problems, to store the evaluated design spaces, to build a library of solvers and tools for generation of design space, to store the configuration and applicability conditions of any design of experiment, and to efficiently retrieve the configurations/applicability conditions/design spaces of design of experiment. In an embodiment, the sub-system 100 includes a processor (s) 104, communication interface device(s), alternatively referred as input/output (I/O) interface(s) 106, and one or more data storage devices or a memory 102 operatively coupled to the processor (s) 104. In an embodiment, the processor (s) 104, can be one or more hardware processors (104). In an embodiment, the one or more hardware processors (104) can be implemented as one or more microprocessors, microcomputers, microcontrollers, digital signal processors, central processing units, state machines, logic circuitries, and/or any devices that manipulate signals based on operational instructions. Among other capabilities, the processor(s) 104 is configured to fetch and execute computer-readable instructions stored in the memory 102. In an embodiment, the sub-system 100 can be implemented in a variety of computing systems, such as laptop computers, notebooks, hand-held devices, workstations, mainframe computers, servers, a network cloud and the like.

The I/O interface(s) 106 can include a variety of software and hardware interfaces, for example, a web interface, a Graphical User Interface (GUI), and the like and can facilitate multiple communications within a wide variety of networks N/W and protocol types, including wired networks, for example, LAN, cable, etc., and wireless networks, such as WLAN, cellular, or satellite. In an embodiment, the I/O interface (s) 106 can include one or more ports for connecting a number of devices to one another or to another server. For example, the I/O interface 106 enables the authorized user to access the system disclosed herein through the GUI and communicate with one or more other similar sub-systems 100.

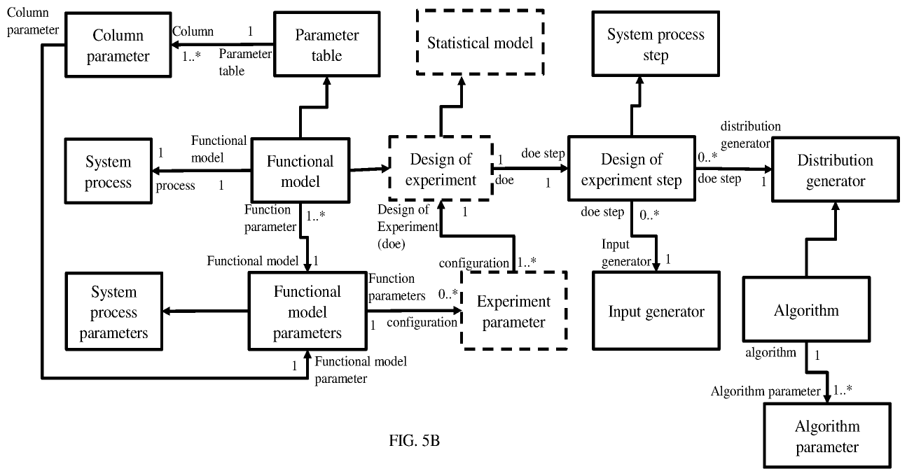

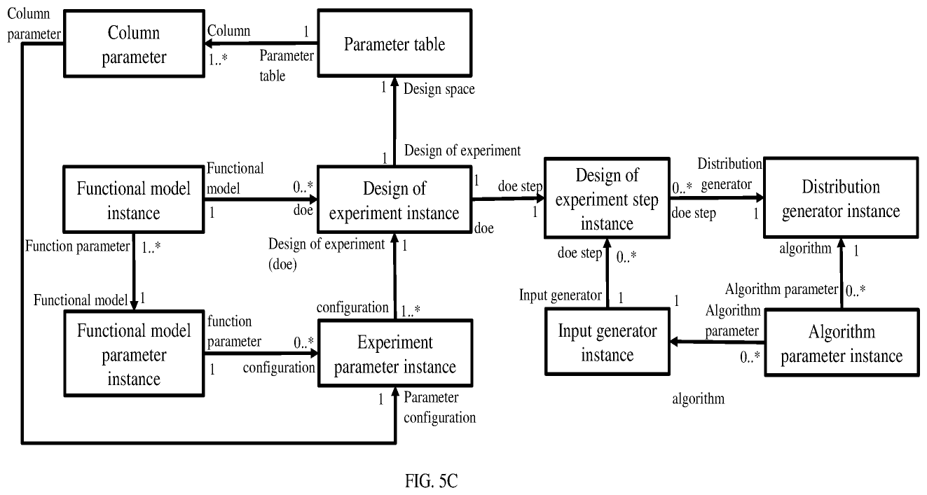

The memory 102 may include any computer-readable medium known in the art including, for example, volatile memory, such as static random access memory (SRAM) and dynamic random access memory (DRAM), and/or non-volatile memory, such as read only memory (ROM), erasable programmable ROM, flash memories, hard disks, optical disks, and magnetic tapes. Thus, the memory 102 may comprise information pertaining to input(s)/output(s) of each step performed by the processor(s) 104 of the sub-system 100 and methods of the present disclosure. In an embodiment, the memory 102 stores a data model that is being used by the sub-system 100 for designing and executing the experiment. Examples of structure of the data model are depicted in FIG. 5A through 5C. In an embodiment, the data model in FIG. 5A through 5C form the computer-readable instructions that are executed by the processors 104 to define and execute the DOE to generate results. Various steps involved in the process of defining and executing the DOE are depicted in FIG. 2 through FIG. 4B and are explained below with reference to the components of the system sub-100.

FIG. 2 is a high-level flow diagram illustrating steps involved in the process of design of experiments, by the sub-system of FIG. 1, according to some embodiments of the present disclosure. In order to enable a model driven system (referred to as “legacy system”) to perform the design of experiments to generate one or more designs, and to execute the one or more designs to generate corresponding result(s), the sub-system 100 can be connected to the legacy system in a suitable manner (for example, in a plug&play manner), and the data-model in the sub-system 100 performs at least part of the data processing associated with/involved in the design of the experiments, and in execution of the design(s) generated as a result of the DOE. The sub-system 100 defines (202) design of experiment (DOE), and then executes (204) the design of experiment. The sub-system 100 uses the data model in FIG. 5A to generate the DOE, by executing the steps in method 300. Different components of the data model are Functions, Statistical model, Step, and Algorithm, and are explained below:

Functions:
In any design of experiment, first and foremost need is to capture the experiment to be perturbed. The utility of ‘Function’ component is to convert a plurality of existing system's executable procedures into a well-defined function on which the perturbation and analysis can be performed.

Statistical Model:
Role of the statistical model component is to capture the configuration, applicability conditions and state of the design as well as co-ordination between all modules of the system sub-100. The statistical model also captures resultant design space(s) after execution of the DOE and provides the design space(s) for further usage and analysis.

Step:
The ‘step’ component handles life-cycle of the sub-system 100. When the sub-system 100 is connected with one or more external systems (also referred to as ‘host systems’) to perform the defining and execution of the DOE, the host system interacts with the ‘step’ component to control the execution of the DOE and to fetch the design space(s).

Algorithm:
The ‘algorithm’ component maintains a repository of procedures (covered in FIG. 2 through FIG. 4B) to help in the execution of design of experiments, namely input set generators and distribution generators. The algorithm component can be selected while configuring the DOE or can be decided while executing the DOE based on certain configuration factors.

FIG. 5B and FIG. 5C depict detailed view the model in FIG. 5A. The data models may be used by the sub-system 100 for designing and executing the DOE. The model components depicted in FIG. 5B and FIG. 5C are required for the sub-system 100 to perform the design of experiments to generate one or more designs, and to execute the generated one or more designs to further generate result corresponding to each of the one or more designs by executing the designs. However, when the sub-system 100 is used to enable a model-driven system to perform the design and execution of designs, the model-driven system (alternately referred to as ‘legacy system’) may also possess one or more of the data components that are required to perform the designing and execution of the designs. In such scenarios, upon determining that the legacy system has one or more of the data components (referred to as “legacy components”) that are required for design and execution of experiments, the sub-system 100 may opt to re-use the legacy components, and other data model components that are required for the data processing, and are not present in the legacy system are used from the data model in the sub-system 100. In another embodiment, the sub-system 100 may determine the legacy components to use, based on a user selection received as input. By providing the data model components and data processing capabilities that the legacy system do not possess, the sub-system 100 enables the legacy system to perform designing and execution of the experiments. However if the legacy system does not have any of the data model components, or if the user opts not to reuse the legacy components for the data processing, the sub-system 100 may function as a stand-alone system, that performs the data processing using the data models in FIG. 5B and FIG. 5C for designing and executing the DOE. For the purpose of explaining how the sub-system 100 enables the legacy system, certain components of the data models explained below are referred to as the legacy components, and the remaining components are referred to as the sub-system components. When the first level model (as in FIG. 5B) is used for defining and executing the DOE, the system 100 uses legacy components ‘system process’, ‘system process parameter’, and ‘system process step’ along with components of the first level model. The first level model includes a functional model, a functional model parameter, a design of experiment, a statistical model, an experiment parameter, a design of experiment step, an algorithm, algorithm parameter, an input generator, a distribution generator, a functional model instance, a functional model parameter instance, a design of experiment instance, an experiment parameter instance, a parameter table, a column parameter, a design of experiment step instance, an input generator instance, a distribution generator instance, and an algorithm parameter instance. Each component of the first level model is explained below (It is to be noted that some of the components are labeled as ‘legacy components’ and some other components are labeled as ‘sub-system components’. The legacy components are components of the model-driven system the sub-system 100 is connected to, so as to perform the design and execution of DOE. The sub-system components are components of the sub-system 100, which may be implementation of/stored in one or more components of the sub-system 100 depicted in FIG. 1):

System Process (Legacy Component):

Any Procedure with determined Input and Output can be treated as a process, and the process block represents any such blocks available in the system's ontology.

System Process Parameter (Legacy Component):

Every system process has corresponding input and outputs defined in the system ontology, and the system process parameter block is used to represent such components.

System Process Step (Legacy Component):

The system process step block is to represent trigger blocks in the existing system. Trigger blocks are the blocks which are used to trigger specific step executions in any system. For instance, in any Business Process Model and Notification (BPMN) Process, every task can be treated as a trigger block.

Functional Model (Sub-System Component):

The functional model block is to capture the definition of function on which the perturbation and analysis is to be performed. The functional model block is associated with existing systems process, and every process has one and only one function counter-part in the sub-system. A Functional Model can form multiple design of experiment definition. A Functional Model can have multiple function parameters, but can have at least 1 function parameter.

Functional Model Parameter (Sub-System Component):

This block is to capture the input and output parameters of the function which is captured in the functional model. The functional model parameter block is associated with existing system's process parameters, and every process parameter has one and only one function parameter counter-part in the sub-system. This parameter is classified as Input and Output using a ‘type’ attribute in the block. A functional model parameter has only one functional model, and can form multiple design of experiment's parameter definition.

Design of Experiment (Sub-System Component):

The ‘Design of Experiment’ block represents definition of Design of Experiment and captures the function that needs to be perturbed by an association to functional model, and also captures the function parameters and their configuration by associating to the experiment parameter. This block also has an association to design process step to enable it the control of design of experiment life-cycle. A Design of Experiment has only one functional model. Further, a design of experiment can have multiple function parameter configuration, and at least one functional parameter configuration. Every Design of experiment is linked to one design of experiment step to maintain the life-cycle of design of experiment.

Statistical Model (Sub-System Component):

The statistical model is a super-class of design of experiment, and is generalized to accommodate all the type of statistical models that may be catered by the sub-system.

Experiment Parameter (Sub-System Component):

The experiment parameter block represents the configuration of function parameter in a definition of design of experiment, the configuration containing the applicability condition, allowed tolerance, default values and so on, of the function parameter. The experiment parameter is linked to only one functional model parameter that specifies the function parameter for which configuration is applicable. The experiment parameter has one design of experiment to indicate for which design the configuration is set.

Design of Experiment Step (Sub-System Components):

This block represents a trigger point to handle the life-cycle of design of experiment. The design of experiment step extends the system process step which are essentially trigger blocks in the existing system. This block is linked to only one design of experiment block. The design of experiment step is linked to one input generator that generates the input set for the design of experiment. This may be linked to one distribution generator that generates the noisy set on the input set generated to factor in the noises generated in real world experiments.

Algorithm (Sub-System Component):

Purpose of this element is to capture a library of procedures that can be executed in the existing system to get specific output after giving specific input. Difference between system process and the algorithm is that the state of algorithm is saved with design space, making it an integral part of the sub-system.

Algorithm Parameter (Sub-System Component):

Every algorithm has some parameters that are specific to the definition of the algorithm, which are passed to an algorithm executor (which may be the existing system or any external system).

Input Generator (Sub-System Component):

The input generator is a type of algorithm which generates the input sets on which the function is executed.

Distribution Generator (Sub-System Component):

This is a type of algorithm which will generate the noise sets from the generated input set to factor in the noises generated in real world experiments.

Design of Experiment Model is the first level model which can be used to configure and create design of experiment problem and whenever the execution of design of experiment problem is triggered from the existing system a second level snapshot of the first level model is created to store the run-time values of entities. Every execution has an instance level model associated with it and this model stores the design space generated from the design of experiment execution. The instance level model is depicted in FIG. 5C and the components are explained below:

Functional Model Instance (Sub-System Component):

This entity is to capture the execution level details of function.

Functional Model Parameter Instance (Sub-System Component):

This entity is to capture the execution level details of function parameter.

Design of Experiment Instance (Sub-System Component):

This entity is to capture the execution level details of design of experiment. Being the bridge entity for communication between different modules of sub-system the design space will be associated with this entity.

Experiment Parameter Instance (Sub-System Component):

This entity captures the execution level details of function parameter configuration such as range bounds, standard deviation, and default/constant value of parameter.

Parameter Table (Sub-System Component):

This entity captures the design space information of design of experiment which includes the input and output value of each run of experiment, thus the format for data persistence is preferred to be a table. This entity contains pointer to a data storage structure. Each parameter table must have only one design of experiment instance. Further, each parameter table must have one or more than one column parameter.

Column Parameter (Sub-System Component):

This entity captures the column information (input/output parameters) of a design space. Each column parameter is linked to an experiment parameter instance to specify which function parameter is referred by this column. Also, each column parameter must have only one parameter table.

Design of Experiment Step Instance (Sub-System Component):

This entity captures the execution level details of design of experiment step, each step may have multiple design of experiment step instances (one for each execution).

Input Generator Instance (Sub-System Component):

This entity captures the execution level details of input generator, each input generator having multiple input generator instances (one for each execution).

Distribution Generator Instance (Sub-System Component):

This entity captures the execution level details of distribution generator, each input generator having multiple distribution generator instances (one for each execution, distribution generator instance is not created if no distribution generator is defined in design of experiment).

Algorithm Parameter Instance (Sub-System Component):

This entity captures the value of algorithm parameter.

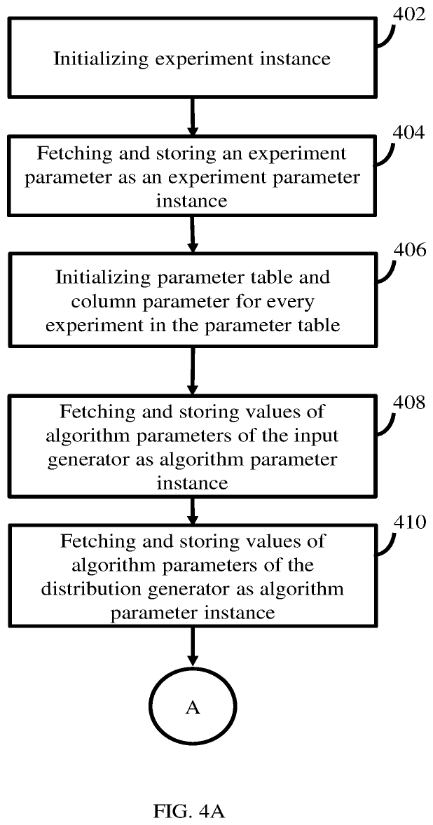

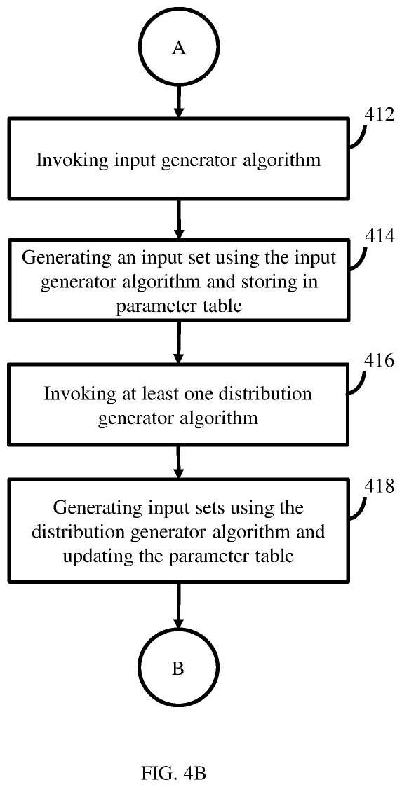

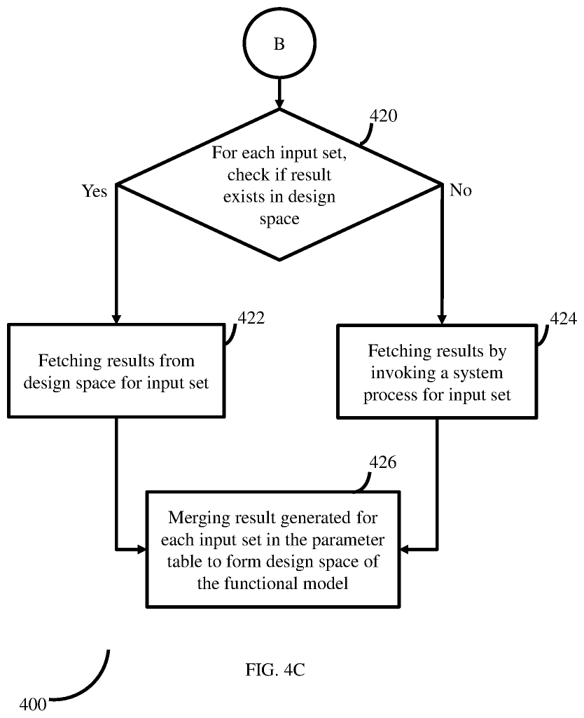

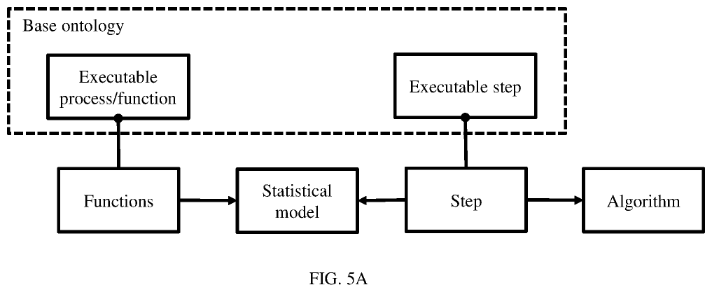

After defining the DOE, the sub-system 100 executes (204) the defined DOE to generate results, which may be provided to the user using a suitable interface (for example, a visual display) provided by the I/O interface(s) 106. Steps involved in the process of executing the defined design of the experiment is depicted in method 400 (FIG. 4).

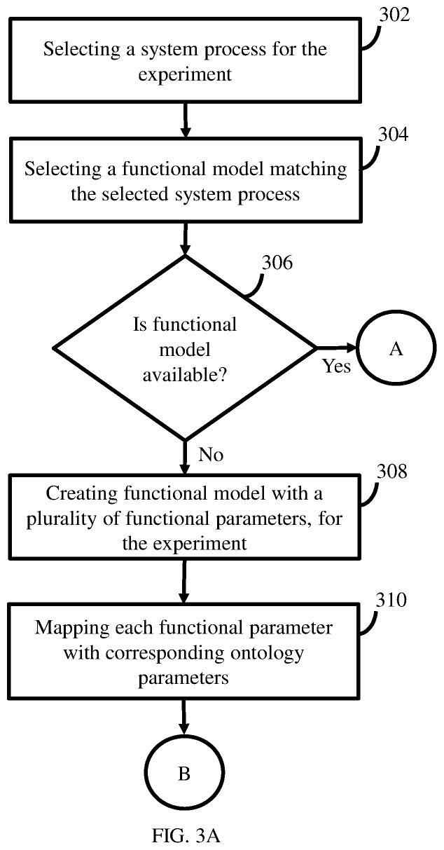

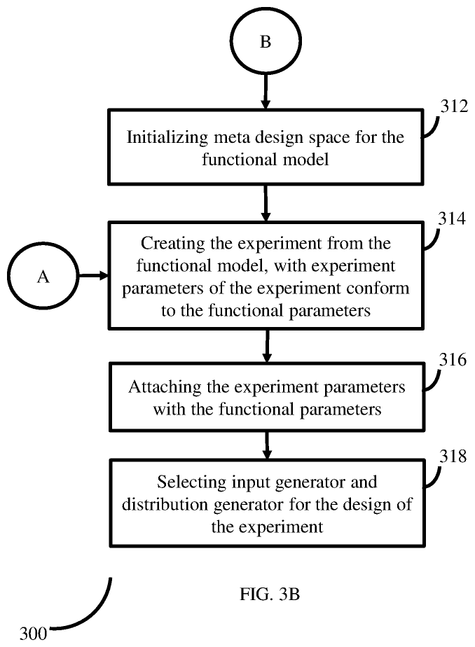

FIG. 3 illustrates a flow diagram depicting steps involved in the process of defining a Design of Experiment (DOE), in accordance with some embodiments of the present disclosure. At step 302, a system process from a plurality of system processes is selected by the system as a candidate for experiment. The selected system process resides in the system's eco-system and the sub-system 100 manages life-cycle of the system process. At step 304, the system selects a functional model from a plurality of functional models stored in the memory 102, as matching the selected system process. The system may select the functional model based on one or more criteria including at least one of a best ontology match approach or based on a user preference collected. The functional model matching the selected system process may or may not exist in the memory 102. If the matching functional model exists, then the sub-system 100 directly executes step 314. If the matching functional model does not exist, then at step 308, the sub-system 100 creates the functional model with a plurality of functional parameters matching a plurality of system process parameters of the selected system process, for the experiment. At step 310, the sub-system 100 maps each functional parameter with corresponding ontology parameters. The created functional parameters exhibit a direct, inclusive, one to one mapping to the system process parameters, thus for any system process parameter in the system (the sub-system 100 is connected to), there is only one functional model parameter in the sub-system 100. Further at step 312, the sub-system 100 initializes meta design space for the functional model. At this stage, the sub-system 100 initializes the parameter table and associates it with the functional model. The sub-system 100 also initializes one and only one column parameter for each functional model parameter of the functional model. The parameter table along with column parameters formulate the schema to store meta design space of respective design of the functional model. At step 314, the sub-system 100 creates the experiment from the functional Model, such that experiment parameters of the experiment conform to the functional model parameters. The sub-system 100 creates the experiment with a plurality of experiment parameters such that the experiment has one and only one functional model associated with it. The experiment parameters conforming to the functional parameters ensures that every experiment parameter has one and only one functional model parameter and that there is an experiment parameter for all the functional model parameters. At step 316, the sub-system 100 attaches each of the experiment parameters with the corresponding functional parameter and provides access to the system enabling it to override the experiment parameter configuration. At step 318, the sub-system 100 selects an input generator and a distribution generator for the design of the experiment. At this step, the sub-system 100 provides a list of input generator and distribution generator algorithms to the system to facilitate the selection of at least one input generator and distribution generator for the experiment. This allows the system to have access and authorization to manage the repository of algorithms in the sub-system 100, which in turn allows the system to create one or more algorithms in the sub-system 100 if they meet a set of input/output specifications of the sub-system 100. The selection of the at least one input generator and distribution generator for the experiment from the list of input generator and distribution generator algorithms is based on at least one criterion configured with the system. For example, the criterion is selection of the at least one input generator and distribution generator may be based on knowledge gained from previous executions, or may be based on a user input dynamically captured by the system. In various embodiments, steps in method 300 may be performed in the same order as depicted in FIG. 3 or in any alternate order that is technically correct. In another embodiment, one or more steps in method 300 may be omitted.

FIG. 4 is a flow diagram depicting steps involved in the process of executing the DOE, according to some embodiments of the present disclosure. At step 402 the sub-system 100 initializes an experiment instance of design of experiment when the execution start, so as to execute multiple design of experiments in parallel. Each execution of the design of experiment has one and only on experiment instance associated with it. Further, at step 404, the sub-system 100 initialize instance for every experiment parameter and associates the experiment parameter instances with the experiment instances i.e. each experiment parameter is fetched and stored as an experiment instance. The experiment parameter instances contain per execution configuration of the experiment parameters. Further, at step 406, the sub-system 100 initializes the parameter table and associates it with experiment instance. The sub-system 100 also initializes one column parameter each for of the experiment parameters of the design of experiment. The parameter table along with the column parameters formulate a schema to store design space of respective design of experiment. At step 408, the sub-system 100 creates instance of the input generator algorithm and algorithm Parameters of the input generator algorithm to enable parallel execution of the input generator algorithms for each DOE execution. At this stage, the algorithm parameters of the input generator are fetched and stored as the algorithm parameter instances. Further at step 410, the sub-system 100 creates instance of the distribution generator algorithm and corresponding algorithm parameters to enable parallel execution of the distribution generator algorithms for each DOE. At this stage, the algorithm parameters of the distribution generator are fetched and stored as the algorithm parameter instances. At step 412, the sub-system invokes the input generation algorithm by providing input generation configuration from the input generation algorithm instance. Once the input generation algorithm is executed, at step 414, the sub-system fetches the generated input sets from output of the input generator algorithm, and stores the generated input sets in the parameter table. Further, at step 416, the sub-system invokes the distribution generator algorithm by providing the generated input set from the parameter table and distribution generation configuration from distribution generator algorithm instance. Once the execution of the distribution generator algorithm is completed, at step 418, the sub-system 100 fetches the generated noisy input sets from the distribution generator algorithm output and merge the generated noise input sets into the parameter table. Once the sub-system 100 formulates the complete list of input sets to be processed, the sub-system 100, at step 420, starts processing every input set, both generated and noisy, and checks if the input set already exists in the design space of the functional model or not. The design space of the functional model stores output for each input set stored. If the input set is available in the design space of the functional model, the sub-system 100, at step 422, fetches corresponding output from the design space of functional model as result and uses the captured output to formulate a design space tuple. If the input set is not available in the design space of functional model, at step 424 the sub-system 100 instructs the system to execute the process with the input set parameter values and then fetches the results from the system post completion of execution of the system process. The sub-system 100 uses the output to formulate a design space tuple. Further, at step 426, each of the design space tuple/record is merged in the existing design space of the functional model to formulate a complete design space of functional model.

### Use-Case Scenario:

Problem (which is Given as Input to the System):

Consider a rectangular cantilever beam (a beam mounted on support from only one side), now when a perpendicular force is applied on the other side, the beam tends to deflect, this deflection is a factor of various parameters such as geometric dimensions of beam, material composition of beam, material properties of beam. By doing Design of Experiment on the respective problem a Design Space for Beam deflection behavior can be obtained.

### Step 1: Defining Design of Experiment

a) System Process and System Process Parameters

The process for calculation of deflection is a function of x₁, x₂ . . . xₙ, where x₁ . . . xₙ can be geometric parameters of beam and material properties of beam, results in y which is beam deflection, thus the process can be represented as:

y=F(l,w,h,t) (1)

where y=beam deflection, l=length of beam, w=width of beam, h=height of beam, t=tensile strength of beam material. Here, F( ) is the system process and l, w, h, t are the system process parameters.

b) Identification and Creation of Functional Model and Functional Model Parameters

The sub-system 100 provides a list of possible functional models to the system and then the system may select one of the functional models from the list or the system may ask the sub-system 100 to create a new functional model. The sub-system 100 creates a functional model F^{fm} and a functional model parameter for each system process parameter l^{fm}, w^{fm}, h^{fm}, t^{fm}, y^{fm}. The sub-system 100 also creates a parameter table PT^{fm} to store the design space of the functional model and column parameters for each functional model parameter.

c) Creation of Design of Experiment and Experiment Parameters

To perform a DOE on a closed range of process parameter values, certain configurations such as range bounds for each process parameter under which the parameters will fluctuate are to be provided. For example, the DOE is performed when length does not exceed 35 cm and the material tensile strength can not exceed 500 psi, thus the sub-system 100 can create an experiment with respective range for experiment parameters. The DOE may have to be performed on the functional model within a closed range of process parameter values, thus for each such DOE, the sub-system 100 creates an experiment, F^{ex}, for the functional model and experiment parameters for each functional model parameter, l^{ex}, w^{ex}, h^{ex}, t^{ex}, y^{ex} that contain the configuration of process parameters.

d) Input Generator and Distribution Generator Configuration

The function of Input Generator is to generate possible input sets for design of experiment that adheres to experiment parameter configuration, such as:

TABLE 1

| L | W | H | T |
|---|---|---|---|
| 32 cm | 20 cm | 5 cm | 450 psi |
| 31 cm | 21 cm | 4 cm | 550 psi |
| 38 cm | 17 cm | 6 cm | 412 psi |

The function of Distribution Generator is to generate possible noisy sets for each individual input set adhering to experiment parameter configuration, such as:

TABLE 2

| L | W | H | T |
|---|---|---|---|
| 32 cm | 20 cm | 5 cm | 450 psi |
| 32.1 cm | 20.2 cm | 4.9 cm | 455 psi |
| 31.8 cm | 19.7 cm | 5.2 cm | 447 psi |
| 31 cm | 21 cm | 4 cm | 550 psi |
| 31.1 cm | 21.8 cm | 4.3 cm | 551 psi |
| 30.8 cm | 21.2 cm | 3.9 cm | 548 psi |
| 38 cm | 17 cm | 6 cm | 412 psi |
| 38.2 cm | 17.2 cm | 6.1 cm | 414 psi |
| 37.8 cm | 16.7 cm | 5.8 cm | 410 psi |

The sub-system 100 provides a list of possible input generators and distribution generators for the system to pick for the given experiment. Once the System picks the appropriate input generator and distribution generator, the sub-system 100 configures the input and distribution generator with the experiment. The sub-system 100 may be configured to allows the system to manage the input generator and distribution generator repositories.

### Step 2: Executing Design of Experiment

Once the design of experiment is defined in the sub-system 100, the system can perform multiple simultaneous execution of the defined design of experiment. Thus, once the design of experiment for the Beam Deflection is created within the above-specified bounds, execution of the Design of Experiment can be started by requesting the sub-system 100 to invoke the execution.

a) Creation of Design of Experiment Instance

The sub-system 100 creates an experiment instance and the experiment parameter instances that contain the state of the current execution of the design of experiment. The sub-system 100 also initializes the parameter table, PT^{exp}, along with the column parameters to store the design space of the current execution of the design of experiment. The sub-system 100 then initializes the input generation instances and distribution generation instances.

b) Generation of Input Sets and Noisy Sets

The sub-system 100 invokes the input generation algorithm using the configuration from input generation instance and fetches the generated individual input set to perform the design of experiment and on each generated individual input set the sub-system 100 performs distribution generation using the distribution generation algorithm and its configuration in the distribution generation instance. The sub-system 100 collects both generated individual input sets and noisy sets and persist them in the PT^{exp}.

c) Creation of Design Space

The sub-system 100 iterates over the generated input sets and checks if the individual input set exists in the design space of functional model PT^{fm}, if it exists the individual output set is picked up from the design space of functional model. If the input set doesn't exist, the sub-system 100 requests the system to invoke the system process with individual input set values as input and the individual output set is picked up from the result of execution and pushed to the design space of functional model with corresponding individual input set and then this individual output set is appended to design space of experiment PT^{exp}. After exhausting complete input sets, the results stored in PT^{exp} are depicted as:

TABLE 3

| L | W | H | T | Y (deflection) |
|---|---|---|---|---|
| 32 cm | 20 cm | 5 cm | 450 psi | 0.5 cm |
| 32.1 cm | 20.2 cm | 4.9 cm | 455 psi | 0.51 cm |
| 31.8 cm | 19.7 cm | 5.2 cm | 447 psi | 0.55 cm |
| 31 cm | 21 cm | 4 cm | 550 psi | 0.61 cm |
| 31.1 cm | 21.8 cm | 4.3 cm | 551 psi | 0.595 cm |
| 30.8 cm | 21.2 cm | 3.9 cm | 548 psi | 0.62 cm |
| 38 cm | 17 cm | 6 cm | 412 psi | 0.4 cm |
| 38.2 cm | 17.2 cm | 6.1 cm | 414 psi | 0.42 cm |
| 37.8 cm | 16.7 cm | 5.8 cm | 410 psi | 0.38 cm |

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments of present disclosure herein address unresolved problem of design of experiments and execution of the design of experiments. The embodiment thus provides a sub-system that can be plugged-into a model-driven system having no capability of designing and executing experiments, to enable the system to perform the designing and execution of experiments.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g. any kind of computer like a server or a personal computer, or the like, or any combination thereof. The device may also include means which could be e.g. hardware means like e.g. an application-specific integrated circuit (ASIC), a field-programmable gate array (FPGA), or a combination of hardware and software means, e.g. an ASIC and an FPGA, or at least one microprocessor and at least one memory with software processing components located therein. Thus, the means can include both hardware means and software means. The method embodiments described herein could be implemented in hardware and software. The device may also include software means. Alternatively, the embodiments may be implemented on different hardware devices, e.g. using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

1. A model driven sub-system (100) for design and execution of experiments consisting of a digital workflow with one or more in-silico experiments in a model-driven system, comprising:
one or more hardware processors (104);
one or more communication interfaces (106); and
one or more memory (102) storing a plurality of instructions, wherein the plurality of instructions when executed cause the one or more hardware processors (104) to:
define design of an experiment, comprising:
selecting a system process for the experiment;
creating a functional model for the selected system process if the functional model does not already exist;
mapping each functional parameter in the functional model with corresponding ontology parameters;
initializing a meta-design space for the functional model;
creating the experiment from the functional model, wherein a plurality of experiment parameters of the experiment conform to the functional parameters of the functional model;
attaching the experiment parameters with the functional parameters; and
selecting an input generator and a distribution generator for the design of the experiment; and
generate a result for the defined design of the experiment, by executing the design of the experiment.

2. The sub-system (100) as claimed in claim 1, wherein executing the defined design of experiment by the system comprising:
initializing an experimental instance of the defined design of experiment;
fetching and storing an experiment parameter as an experiment parameter instance;
initializing a parameter table that stores the defined design of experiment, and a column parameter for every experiment in the parameter table;
fetching and storing values of a plurality of algorithm parameters of the at least one input generator, as an algorithm parameter instance;
fetching and storing values of a plurality of algorithm parameters of the at least one distribution generator, as the algorithm parameter instance;
invoking at least one input generator algorithm by feeding an input for the at least one input generator algorithm;
generating an input set using the at least one input generator algorithm and storing the input set in the parameter table;
invoking at least one distribution generator algorithm by feeding one or more inputs for the at least one distribution generator algorithm;
generating input sets using the distribution generator algorithm and updating the parameter table using the generated input sets;
determining whether result for each input set exists in the meta-design space;
fetching results for each of the input sets, from the meta-design space, if the result already exists;
fetching results for each of the input sets, by invoking a system process for the input set, if the result does not exist in the meta-design space; and
merging the result generated for each of the input sets in the parameter table to form a complete design space of the functional model.

3. A processor implemented method (200) for design and execution of experiments consisting of a digital workflow with one or more in-silico experiments in a model-driven system, the method comprising:
defining (202) design of an experiment, via one or more hardware processors (104), comprising:
selecting (302) a system process for the experiment;
creating (308) a functional model for the selected system process if the functional model does not already exist;
mapping (310) each functional parameter in the functional model with corresponding ontology parameters;
initializing (312) a meta-design space for the functional model;
creating (314) the experiment from the functional model, wherein a plurality of experiment parameters of the experiment conform to the functional parameters of the functional model;
attaching (316) the experiment parameters with the functional parameters; and
selecting (318) an input generator and a distribution generator for the design of the experiment; and
generating a result for the defined design of the experiment, by executing (204) the design of the experiment, via the one or more hardware processors (104).

4. The processor implemented method as claimed in claim 3, wherein executing the defined design of experiment comprising:
initializing (402) an experimental instance of the defined design of experiment;
fetching and storing (404) an experiment parameter as an experiment parameter instance;
initializing (406) a parameter table that stores the defined design of experiment, and a column parameter for every experiment in the parameter table;
fetching and storing (408) values of a plurality of algorithm parameters of the at least one input generator, as an algorithm parameter instance;
fetching and storing (410) values of a plurality of algorithm parameters of the at least one distribution generator, as the algorithm parameter instance;
invoking (412) at least one input generator algorithm by feeding an input for the at least one input generator algorithm;
generating (414) an input set using the at least one input generator algorithm and storing the input set in the parameter table;
invoking (416) at least one distribution generator algorithm by feeding one or more inputs for the at least one distribution generator algorithm;
generating (418) input sets using the distribution generator algorithm and updating the parameter table using the generated input sets;
determining (420) whether result for each input set exists in the meta-design space;
fetching (422) results for each of the input sets, from the meta-design space, if the result already exists;
fetching (424) results for each of the input sets, by invoking a system process for the input set, if the result does not exist in the meta-design space; and
merging (426) the result generated for each of the input sets in the parameter table to form a complete design space of the functional model.

5. A computer program product comprising a non-transitory computer readable medium having a computer readable program embodied therein, wherein the computer readable program, when executed on a computing device, causes the computing device to perform:
defining design of an experiment, comprising:
selecting a system process for the experiment;
creating a functional model for the selected system process if the functional model does not already exist;
mapping each functional parameter in the functional model with corresponding ontology parameters;
initializing a meta-design space for the functional model;
creating the experiment from the functional model, wherein a plurality of experiment parameters of the experiment conform to the functional parameters of the functional model;
attaching the experiment parameters with the functional parameters; and
selecting an input generator and a distribution generator for the design of the experiment; and
generating a result for the defined design of the experiment, by executing the design of the experiment.

6. The computer program product as claimed in claim 5, wherein executing the defined design of experiment by the system comprising:
initializing an experimental instance of the defined design of experiment;
fetching and storing an experiment parameter as an experiment parameter instance;
initializing a parameter table that stores the defined design of experiment, and a column parameter for every experiment in the parameter table;
fetching and storing values of a plurality of algorithm parameters of the at least one input generator, as an algorithm parameter instance;
fetching and storing values of a plurality of algorithm parameters of the at least one distribution generator, as the algorithm parameter instance;
invoking at least one input generator algorithm by feeding an input for the at least one input generator algorithm;
generating an input set using the at least one input generator algorithm and storing the input set in the parameter table;
invoking at least one distribution generator algorithm by feeding one or more inputs for the at least one distribution generator algorithm;
generating input sets using the distribution generator algorithm and updating the parameter table using the generated input sets;
determining whether result for each input set exists in the meta-design space;
fetching results for each of the input sets, from the meta-design space, if the result already exists;
fetching results for each of the input sets, by invoking a system process for the input set, if the result does not exist in the meta-design space; and
merging the result generated for each of the input sets in the parameter table to form a complete design space of the functional model.
